SEQUENCE LISTING (23 sequences)

SEQ ID NO: 1 - DNA, 10 bp, Artificial Sequence
  misc_feature (3)..(3): any nucleotide
tcnggnnnnn

SEQ ID NO: 2 - DNA, 10 bp, Artificial Sequence
  misc_feature (3)..(3): any nucleotide
ccnggnnnnn

SEQ ID NO: 3 - DNA, 10 bp, Artificial Sequence
  misc_feature (1)..(1): any nucleotide
nnnnnccnga

SEQ ID NO: 4 - DNA, 10 bp, Artificial Sequence
  misc_feature (1)..(1): any nucleotide
nnnnnccngg

SEQ ID NO: 5 - DNA, 10 bp, Artificial Sequence
  misc_feature (3)..(3): any nucleotide
gcnggnnnnn

SEQ ID NO: 6 - DNA, 10 bp, Artificial Sequence
  misc_feature (3)..(3): any nucleotide
acnggnnnnn

SEQ ID NO: 7 - DNA, 10 bp, Artificial Sequence
  misc_feature (1)..(1): any nucleotide
nnnnnccngc

SEQ ID NO: 8 - DNA, 10 bp, Artificial Sequence
  misc_feature (1)..(1): any nucleotide
nnnnnccngt

SEQ ID NO: 9 - DNA, 24 bp, Artificial Sequence
  Synthetic
gcgtaatacgactcactatggacg

SEQ ID NO: 10 - DNA, 36 bp, Artificial Sequence
  Synthetic
gtcttcgtgtaacgtccatagtgagtcgtattacgc

SEQ ID NO: 11 - DNA, 36 bp, Artificial Sequence
  Synthetic
gtcttcgtgtggcgtccatagtgagtcgtattacgc

SEQ ID NO: 12 - DNA, 36 bp, Artificial Sequence
  Synthetic
gtcttcgtgtttcgtccatagtgagtcgtattacgc

SEQ ID NO: 13 - DNA, 36 bp, Artificial Sequence
  Synthetic
gtcttcgtgtcccgtccatagtgagtcgtattacgc

SEQ ID NO: 14 - DNA, 24 bp, Artificial Sequence
  Synthetic
gcgtaatacgactcactatggacg

SEQ ID NO: 15 - DNA, 36 bp, Artificial Sequence
  Synthetic
gtcttcgtgtcacgtccatagtgagtcgtattacgc

SEQ ID NO: 16 - DNA, 10 bp, Artificial Sequence
  Synthetic
attgtccgcg

SEQ ID NO: 17 - DNA, 11 bp, Artificial Sequence
  misc_feature (7)..(7): a nonstandard nucleotide of the instant invention
attgtcnnnng

SEQ ID NO: 18 - DNA, 14 bp, Artificial Sequence
  Synthetic
tcacagagagagca

SEQ ID NO: 19 - DNA, 14 bp, Artificial Sequence
  misc_feature (6)..(6): any nucleotide
tcacannnnnnnna

SEQ ID NO: 20 - DNA, 25 bp, Artificial Sequence
  Synthetic
attgtccgcggtcacagagagagca

SEQ ID NO: 21 - DNA, 25 bp, Artificial Sequence
  misc_feature (17)..(17): a nonstandard nucleotide of the instant invention
attgtccgcggtcacannnnnnnna

SEQ ID NO: 22 - DNA, 25 bp, Artificial Sequence
  misc_feature (7)..(7): a nonstandard nucleotide of the instant invention
attgtcnnnngtcacannnnnnnna

SEQ ID NO: 23 - DNA, 52 bp, Artificial Sequence
  Synthetic
ctgagaccatggtattgatccacaacttgctctctctgtgaccgcggacaat

(19) United States
(12) Patent Application Publication
(10) Pub. No.: US 2009/0270601 A1
(43) Pub. Date: Oct. 29, 2009

(54) Differential detection of single nucleotide polymorphisms

(76) Inventors: Steven Albert Benner, Gainesville, FL (US); Shuichi Hoshika, Gainesville, FL (US); Nicole Aurora Leal, Gainesville, FL (US)

Correspondence Address: Steven A. Benner, 1501 NW 68th Terrace, Gainesville, FL, 32605-4147, US

(21) Appl. No.: 12/386,595

(22) Filed: Apr. 21, 2009

Related U.S. Application Data
(60) Provisional application No. 61/124,961, filed on Apr. 21, 2008.

(51) Int. Cl. C12P 19/34; C07H 21/04

(52) U.S. Cl. 536/23.1; 435/91.2

(57) ABSTRACT

This application claims processes and compositions that enable discovery of single nucleotide polymorphisms (SNPs) and other sequence variation that follows two essentially identical sequences, one a reference, the other a target, as well as SNPs discovered using these processes and compositions. The inventive process comprises preparation of four sets of primers, “T-extendable”, “A-extendable”, “C-extendable”, and “G-extendable”. These primers, when templated on a reference genome, add (respectively) T, A, C, and G to their 3′-ends. The invention also comprises a step where these primer sets are separately bound to complementary sequences on target DNA and, once bound, prime extension reactions using target DNA as the template. If the target DNA directs incorporation of the same nucleotide as the reference DNA, then the T-, A-, C-, and G-extendable primers are extended (respectively) by T, A, C, and G. The architecture of the process distinguishes products from these extensions from products derived if not T, not A, not C and not G (“3N” or “3”, to indicate the other three nucleotides) are not added. Thus, this process discovers differences between the target and reference DNA in the site queried by the primer extension reaction. The distinction makes the two kinds of products either separable or differentially extendable. This distinction is used to disregard products that added T, A, C, and G and to identify the sequence(s) of primers that added not-T, not-A, not-C, and not-G. Further and optionally, information from these sequences identifies loci of the SNPs in an in silico genome.

### CROSS REFERENCE TO RELATED APPLICATIONS

This application claims priority to U.S. Provisional Patent Application 61/124,961, filed Apr. 21, 2008.

### FIELD OF THE INVENTION

This invention relates generally to processes and compositions for analyzing DNA sequences and more particularly to methods and compositions for discovering single nucleotide variations, or “polymorphisms”, sites in a target sequence of DNA that hold a nucleotide that is different from the nucleotide in the analogous site in an analogous reference sequence. This invention also relates to SNPs discovered using the processes and compositions of the instant invention.

### BACKGROUND

Genetic variation distinguishing the genomes of individuals within a species of organisms is a major, if not the major, determinant of the differential responses of those individuals to different environments, their differential susceptibility to disease, and (in medicine, human or animal) their differential response to various therapeutic regimens. Accordingly, discovering genetic differences (such as “single nucleotide polymorphisms”, or SNPs) between different individuals, between tissues within an individual (such as those that arise in cancer tissues), or even between analogous sites in chromosomes in a diploid individual (which shows the differences in the genetic material received from the two parents) is a major goal of research in many laboratories. SNP discovery and detection is therefore emerging as a major theme in research on many species (including bacteria, animals, fungi, and plants), and in human and animal medicine. Direct evidence for the utility of any tools that discover or detect variation of this type is the number of National Institutes of Health (NIH) opportunities for funding research to develop such tools (for example RFA-HL-08-004).

“SNP discovery” is fundamentally a different problem from “SNP detection”. The second presumes that one already knows the variant sequence that one wishes to detect. Knowing what one wants to find makes finding it easier to find it, of course, and many tools are available for identifying known single nucleotide polymorphisms (SNPs) in a sample of DNA [Sjo08] [Kim08]. In contrast, very few tools exist for the high-throughout discovery of unknown genetic variations.

Many approaches in the art to discover SNPs simply do standard DNA sequencing on the genomes (or parts of genomes) of many individuals. We call these “brute force” approaches”. For example, the combined work of the SNP Consortium [Sac01] and other public projects has discovered ˜10 million SNPs in various human genomes just by sequencing. The work continues in an NIH program to re-sequence many different cancer tissues, hoping that variation between cell types (cancerous, non-cancerous) that is significant to the cancer disease is not lost amid irrelevant variation arising from the “mutator phenotype” of cancer cells.

A non-brute force approach for discovering single nucleotide differences that distinguish a target genome from a reference genome is the cell-based approach described by Faham et al. [Fah01] [Fah05] (the terms “target” and “reference” will be used throughout this disclosure; the distinction is theoretically arbitrary, but is needed in the context of descriptions of specific architectures). This approach exploits the mismatch repair system in vivo in E. coli. Mismatch repair detection (MRD) was used [Fak04] in the search for SNPs that separate cancer cell genomes from the genomes in their untransformed counterparts [Pet07]. Here, the technique permitted a search limited to 10.3 Mb (ca. 0.3%) of the tumor genome, or ca. 8.5 Mb of protein coding sequence. Approximately 90% of the amplicons screened showed a perfect match to the reference genome sequence. An additional 8.7% of the amplicons had variations that distinguished them from the corresponding matched normal samples, suggesting these were likely germ line variations. These were also removed from subsequent analysis. The remaining 0.3% of amplicons were sequenced to discover 54 putative somatic mutations.

Brute force approaches for SNP discovery in various species are assisted today by the fact that often, a whole genome sequence for an individual of that species has been determined and is recorded in a computer database (an in silico genome). For humans, this is the case as well. In this case, we speak of “re-sequencing”, rather than “de novo sequencing”. Brute force re-sequencing is less expensive than de novo sequencing because without an in silico sequence, short fragments of DNA sequence determined in the sequencing experiments must be assembled into a closed chromosome using only information from other short fragments. In resequencing, fragment assembly is guided by the in silico genome. This is simpler, in the same way as assembling a jigsaw puzzle is simpler when the pieces can be laid on top of a picture of the puzzle.

### BRIEF DESCRIPTION OF THE DRAWINGS

FIG. 1. Schematic of the choices made when designing an architecture to implement the instant invention.

FIG. 2. Schematic showing the generation of an underhang.

FIG. 3. Results from Example 2, part 1. PAGE (16%) showing the incorporation of irreversible and reversible terminators using various templates. Note how in each case, the correct terminated nucleotide is incorporated.

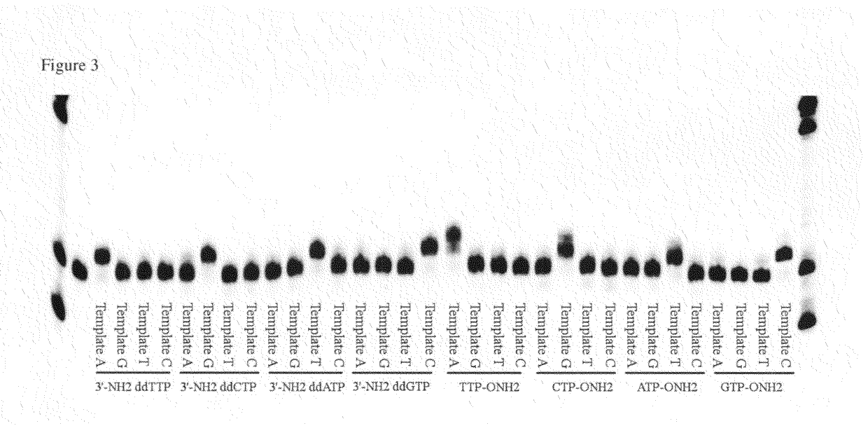

FIG. 4. Results from Example 2, part 2. PAGE (20%) showing incorporation, cleavage and subsequent extension using in competition assays using reversible and irreversible terminators and a template containing A at position N+1. Lane 2: TTP-ONH₂, 3′amino dd C, G and ATP; Lane 5: CTP-ONH₂, 3′amino ddT, G and ATP; Lane 8: GTP-ONH₂, 3′amino dd C, T and ATP; Lane 11: ATP-ONH₂, 3′amino dd G, T, and CTP. Cleavage of primary extension reactions in lanes 3, 6, 9 and 12. Final extension with dNTPs in lanes 4, 7, 10 and 13.

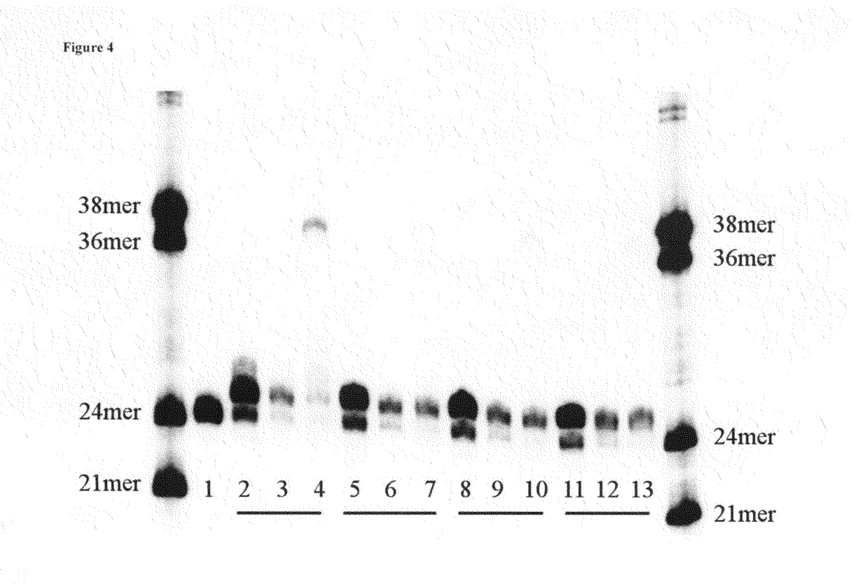

FIG. 5. Ligation in a 11+{5+8*+1} format between standard and SAMRS (indicated by *) fragments, with the products resolved by 20% PAGE. From Example 3.

### DESCRIPTION OF THE INVENTION

### Overview

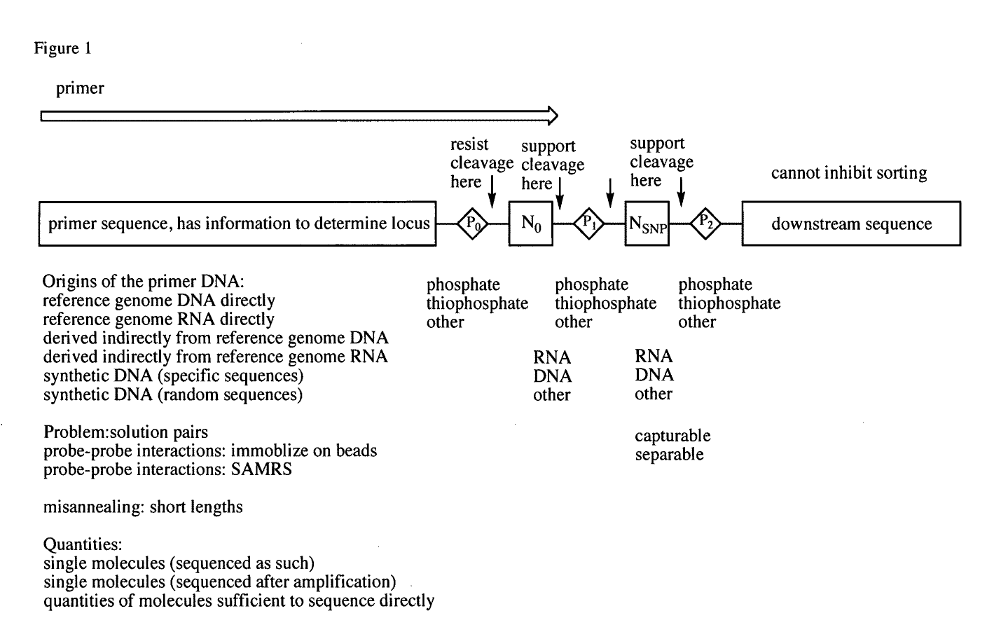

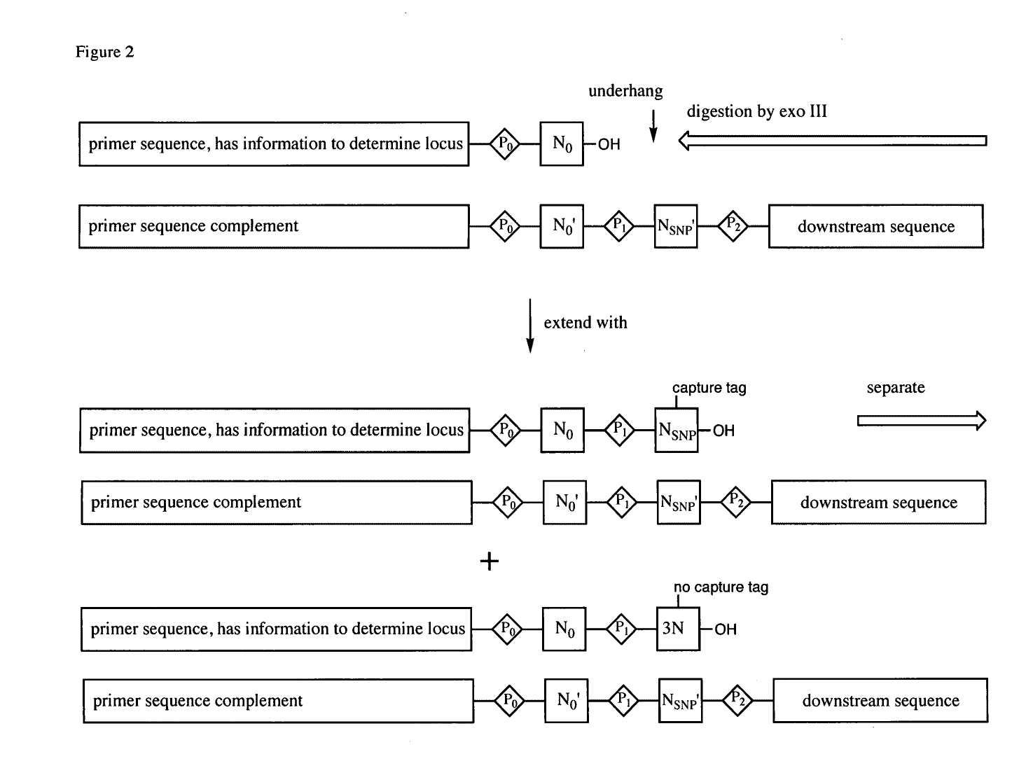

### FIGS. 1, and 2

The instant invention discovers a site or sites (the “queried” site or sites, the site or sites that direct(s) the incorporation of the first nucleotide added to the 3′-end of a primer in a template-directed polymerization process) in a target segment of DNA that differ from that site (or sites) present in a reference segment of DNA. This variation may be a single nucleotide polymorphisms (SNPs), different nucleotides present in analogous sites in two sequences; it may also arise because the directing sequence has been deleted, with the first directing nucleotide coming from a portion of the target sequence that is non-homologous to that portion in the reference sequence.

The process of the instant invention obligatorily comprises four essential steps. The first step provides four sets of primers, which are designated “T-extendable”, “A-extendable”, “C-extendable”, and “G-extendable”. These primers, when targeted against a reference genome as a template, add (respectively) T, A, C, and G to their 3′-ends in a template-directed primer extension reaction.

The second step presents these four primer sets, separately, to a sample of the target DNA. In this presentation, members of each set are contacted to the target DNA in buffer appropriate for them to bind to their complementary segments within the target DNA.

In the third step, bound members of the primer set serve as primers for a template-directed primer extension reaction using the target genome as the template. If the template from the target genome presents the same templating nucleotide for the first nucleotide added in the extension reaction as the reference genome, then the T-extendable, A-extendable, C-extendable, and G-extendable primers will be extended (respectively) by T, A, C, and G. If, however, the template from the target genome presents a nucleotide different from the reference genome, then the T-extendable, A-extendable, C-extendable, and G-extendable primers will be extended (respectively) by not T, not A, not C, and not G (referred to here as “3N” or “3”, to indicate the other three nucleotides, where which of the other three is understood by context). In these cases, the primers have discovered a difference between the target and reference DNA at the queried site.

The architecture of the third step is made such that the T-extendable, A-extendable, C-extendable, and G-extendable primers that add (respectively) not-T, not-A, not-C, and not-G give products that are physically distinct from the products arising when those primers added T, A, C, and G (respectively). Those that added T, A, C, and G (respectively) did not discover variation; they are not necessarily of interest. The primers that added “not-T”, “not-A”, “not-C”, and “not-G” did discover variation; they are of extreme interest. Whether done singly, or when presented as a mixture of extension primers enriched (relative to those primers that did not discover a SNP) in primers that have discovered variation, they are a useful deliverable.

A fourth process, which can be done in various ways, uses this physical distinction between extension products that did discover variation at the queried site from those that did not. As described in detail below, this distinction may render the first products separable from the second; in this case the fourth step involves separation. The distinction may have appended an irreversible terminator, such as a 2′,3′-dideoxynucleotide, to the second, rendering it not clonable or sequencable, while a reversible terminator such as a 2′-deoxy-3′-ONH₂-nucleotide, might be added to the first. In this case, the fourth step involves differential cloning or sequencing.

In either case, it is for most applications desirable to determine the sequence of the primers that have discovered the variation at the queried site. This may be realized by sequencing the primers immediately preceding the added nucleotide. This may be done classically, by cloning, or by one of the next generation sequencing instruments offered by 454, Solexa, Helicos, Intelligent BioSystems, or another organization. The information obtained from this sequencing may allow the identification of the locus of the SNP in an in silico reference genome, for example.

This specification teaches the distinction between the invention and the architecture used to implement the invention. The architecture used to execute this process preferably ensures that the length of the primer is sufficient to carry enough information to identify the locus of the SNP in the corresponding in silico genome, at least in a useful number of cases to a useful degree of uniqueness. This length depends on the nature of the genome being probed. More information is required to locate a SNP in a larger genome than in a smaller one. Further, depending on the nature of the genome being probed, special arrangements are made to handle heterozygosity (in diploid genomes) and the repetitive “low-information content” nature of 90% of the human genome, reference and target.

Many architectures can be used to implement the instant invention. They differ (for example) in the way in which the four primer sets are provided, the way in which the N-extendable primers (where N is used to designate T, A, C, or G) that were extended with not-N are recovered, how the recovered sequences are analyzed, how specific challenges presented by a specific genomes are solved, and the extent to which an architecture trades off coverage (the fraction of variation in a sample of DNA discovered) and cost. Various of these are discussed in the Detailed Description and exemplified in the Examples.

The teachings of this disclosure are inventive in multiple ways. First, they are inventive in the processes that they disclose that use physical DNA from a genome to generate four different primer sets. Also inventive are the processes disclosed that exploit the primer sets. Also inventive are processes that deplete primers that prime against repeats from a downstream deliverable. Another of the inventive teachings of this disclosure is that determining the heterozygosity of a diploid individual provides a substantial sampling of the difference between the genome of the individual and the average genome of a population. A further invention is the variation (and the locus of the associated sites queried) that are derived from the combination of all of these.

### DEFINITIONS

454: The DNA sequencer that uses a strategy based on pyrophosphate sequencing, developed by a Connecticut firm, that implements a SuCRT sequencing architecture.
AEGIS: Artificially Expanded Genetic Information Systems, a kind of DNA that forms Watson-Crick pairs DNA containing complementary AEGIS components, but not natural DNA [Ben04].
Analogous segments: In the comparison of two genomes, we speak of homologous versus analogous segments. Homology is a theoretical term, and refers two segments in two genomes in two organisms that are related by common ancestry. Analogous is an operational term, and refers to two sequences that are largely identical over a significant length.
Architectures: The collection of detailed procedures and protocols that implement the steps in the invention.
DNA fragment: A physical piece of DNA, generally duplex.
DNA fragmentation: Breaking of the physical DNA into pieces. This is done, inter alia, by restriction digestion, sonication, or focused disruption using a Covaris instrument known in the art, most preferably using a Covaris instrument when fragments 50-200 nucleotides are desired.
DNA Segment: Representation of a physical piece of DNA on paper or in a computer.
Explicit chemical synthesis: Phosphoramidite synthesis of specific DNA sequences, under control of software, for example, distinct from the synthesis of sequences as part of library synthesis (e.g., split and pool, or through the addition of phosphoramidite mixtures).
Homologous segments: In comparing two DNA molecules, we speak of homologous versus analogous segments. Homology is a theoretical term, and refers two segments in two genomes in two organisms that are related by common ancestry. Analogous is an operational term, and refers to two sequences that are largely identical over a significant length.
In silico genome: A computerized genome from a computer database, preferably searchable.
Locus: location in a genome.
—ONH₂: A capturable, reversible terminator, an alkyloxylamine, described in U.S. patent application Ser. No. 11/373,415, the disclosure of which is incorporated in its entirety by reference.
Overhang: When reference is made to a 5′- or 3′-end, a single stranded extension preceding or following (respectively) a duplex region.
PEG: Polyethylene glycol.
Physical DNA: This refers to tactics where the DNA or RNA from a reference or target genome directly provides the material for the primer without an intervening in silico analysis, or the chemical synthesis of DNA. The physical DNA can be used directly. Alternatively, the physical DNA can be amplified by growth of the host organism, cloning followed by growth of the clones, or PCR amplification outside of a living cell.
Polishing: This refers to a process of rendering the DNA fragments blunt ended, either by removal of overhangs with nuclease digestion (e.g., with mung bean nuclease or Exo T, sold by New England Biolabs) of the single stranded overhangs and/or underhangs, or by polymerase filling in of 3′-underhangs by treatment with DNA polymerase and 2′-deoxynucleoside triphosphates (a fill in protocol). It is understood that failure to polish the ends of all duplex fragments need not be problematic in a stochastic process.
Polymerase: Includes DNA polymerases and reverse transcriptases.
SAMRS: Self-Avoiding Molecular Recognition System, a kind of DNA that forms Watson-Crick pairs with natural DNA, but not other SAMRS DNA, as described in U.S. patent application Ser. No. 12/229,159, the disclosure of which is incorporated in its entirety by reference.
SNAP2: An architecture where two short fragments are assembled via a dynamic bond on a template under conditions of dynamic equilibrium; these fragments prime synthesis when the bond is formed. This is described in U.S. patent application Ser. No. 11/702,372, the disclosure of which is incorporated in its entirety by reference.
SNP: Single nucleotide polymorphism.
SuCRT: Sequencing using cyclic reversible termination.
Underhang: When reference is made to a 5′- or 3′-end, this indicates that this end is preceded or followed (respectively) by a single stranded region on the complementary DNA.

### 1. Step 1. Generating the Primer Sets

The first step of the process of instant invention provides four sets of primers, which are designated “T-extendable”, “A-extendable”, “C-extendable”, and “G-extendable”. These primers, when targeted against a reference genome as a template, add (respectively) T, A, C, and G to their 3′-ends in a template-directed primer extension reaction.

### 1.1 Generating the Primer Sets by Direct Chemical Synthesis

These sets can, of course, be prepared by standard phosphoramidite-based chemical synthesis, when the sites to be queried in the target DNA are known in the reference DNA. This is the preferred process when a small number (up to 10000) primers are desired. If multiplexed primer extension is desired, SAMRS components are preferably incorporated into the 3′-end of each primer. Most preferably, those primers are 25 nucleotides in length, where the first (from the 5′-end) 16 of these are standard nucleotides, the next 8 are SAMRS nucleotides, and the last nucleotide at the 3′-end is a standard nucleotide. Use of SAMRS makes the primers not interact with other primers. Direct chemical synthesis of the four sets of extendable primers is also preferred when variation is desired in a specific gene, such as the APC gene involved in colon cancer.

Superficially, this approach may resemble the Comparative Genome Sequencing (CGS) offered by NimbleGen. Here, arrays are synthesized to permit brute force re-sequencing (or survey re-sequencing) of entire genomes. This is a brute force approach for identifying the locations of SNPs, insertions, or deletions. It is distinct from the instant invention by not involving the discovery of SNPs through the delivery of mixtures enriched in fragments that contain, or are adjacent to, SNPs. In the NimbleGen approach, both regions that contain SNPs and sequences that do not contain SNPs are re-sequenced.

Alternatively, split-and-pool methods can be used to generate libraries of oligonucleotides supported, for example, on beads. Then, the beads can be sorted based on their ability of the primers that they support to add a T, A, C, and G as the first nucleotide added when templated using the reference genome. Alternatively, the primers on the beads that, when templated using the reference genome, add three of the four standard nucleotides can be irreversibly blocked (using, for example, the 2′,3′-dideoxynucleoside triphosphates for the 3 nucleosides). This is limited by the number of beads that can be conveniently used (for example, a split and pool library that contains all 16-mers on average once requires approximately 4 billion beads). It does not require, however, knowledge of the sequence of the reference genome.

Alternatively, solution-based libraries constructed from random sequences can be prepared, and converted to the primer sets in four separate batches by templating these on multiple exemplars of the reference genome, where nucleotide N is added as the triphosphate at the same time as the triphosphates of the 3N nucleotides are added, where the products arising from the addition of N can be separated from the products arising from the addition of 3N, or where the products arising from the addition of 3N are irreversibly blocked from participating in the cleavage reaction that regenerates the N-extendable primer set, or irreversibly blocked from participating in another downstream process. As is understood by those skilled in the art, this has the advantage of not being limited by the number of beads that can be physically created, or the number of sequences that can be deliberately synthesized on (for example) a two dimensional array. It also does not require knowledge of the sequence of the reference genome.

1.2 Obtaining Primer Sets from the Reference DNA Itself

### 1.2.1 Fragmenting the DNA

In some implementations, and especially when large numbers of extendable primer sets are desired, processes are desired that generate the primer sets using physical reference DNA. These come in two classes, one that uses the physical reference DNA as part of the primers themselves, the other that uses the reference DNA to template the synthesis of the primer sets.

Both architectures require fragmentation of a sample of reference duplex DNA. This can be done by simple sonication to give duplex fragments between 1000 and 10000 nucleotide pairs in length. This will generate a fragment with an end at any particular site with a probability of one in 1000 to one in 10000. Underhung primers are then obtained by exonuclease III digestion, a process well known in the art.

Shorter fragments are preferred, for example, for primer sets to be used with immobilized templates or templates to be used with immobilized primers, or to get sets with more sites queried per unit of DNA absorbance. These are preferably generated by fragmentation using an instrument sold by Covaris, Inc. (14 Gill Street, Unit H Woburn, Mass. 01801-1721). This instrument generates, with narrow length distributions, duplex fragments as short as 50 base pairs or as long as 1000 base pairs. Fragments 50-100 nucleotides in length are presently preferred. Underhangs are then obtained by exonuclease III digestion.

For some of architectures that implement the process of the instant invention, the ends of the fragments are “polished” (rendered to be blunt ended). This is achieved either by removal of overhangs with nuclease digestion (e.g., with mung bean nuclease or Exo T, sold by New England Biolabs) of the single stranded overhangs and/or underhangs, or by polymerase filling in of 3′-underhangs by treatment with DNA polymerase and 2′-deoxynucleoside triphosphates (a fill in protocol). The second is preferred; all are well known in the art. It is understood that failure to polish ends of all duplex fragments need not be problematic in a stochastic process.

The method of obtaining the fragments is not central to the inventive process. Other ways of obtaining fragments, including library synthesis, obtaining them from archival collections, and from restriction digestion (for example) may also be used.

In most applications, the fragments of reference DNA are rendered inactive for subsequent steps. When subsequent steps involve ligation, the 5′-phosphate group is preferably removed by a phosphatase. When ligation and/or primer extension is involved, the 3′-end is blocked by adding a 2′,3′-dideoxynucleotide. This is referred to as “capping”.

### 1.2.2 Ligating Primer DNA on the Fragments of Reference DNA

The capped fragments of melted reference DNA, preferably 100 to 200 nucleotides long (so that self-annealing is slowed) act as templates to ligate fragments of DNA, which may be prepared in any of the ways above. Ligation is especially valuable when SAMRS nucleotides are desired in the 3′-end of the primers, to prevent primer-primer interactions in subsequent steps of the process. In a primer-synthesis architecture involving ligation, fragments targeted either against specific regions of the gene or generated as libraries to cover any sequence. Preferably, the fragments that are to become the 5′-end of the primer are built from standard nucleotides, are 5-20 nucleotides in length, and if prepared as a random library, are most preferably 8-12 nucleotides in length. They lack a 5′-phosphate. Preferably, the fragments that are to become the 3′-end of the primer are built from standard+SAMRS nucleotides, are 5-20 nucleotides in length, and if prepared as a random library, are most preferably 8-12 nucleotides in length, with at least 6 of the last (3′-end) nucleotides being SAMRS. They have a 5′-phosphate. These primers are then separated into “T-extendable”, “A-extendable”, “C-extendable”, and “G-extendable” sets using one of the methods below.

1.2.3 Deriving the Primer Sets from the Physical DNA of the Reference Genome

Alternatively, the DNA from the reference genome can itself physically be incorporated into the primers. A simple approach to generate the four N-extendable primer sets involves treatment of the reference genome with restriction sites that leave a 3′-underhang where the complementary strand (now a 5′-overhang) templates the addition of N (T, A, C or G) as the first nucleotide in the extension reaction. This has the disadvantage of allowing the primer sets to query only those sites where a corresponding restriction enzyme can be found for use. This is, in turn, limited by the fact that most restriction sites that cleave within their recognition region have palindromic recognition sequences.

An alternative approach generates libraries of 3′-underhangs from duplex fragments of the reference DNA. For example, in one such architecture, the reference genome is randomly fragmented to create duplexes. Partial digestion with 3′-exonuclease such as exonuclease III generates a library of underhang duplexes. These are processed as described below.

### 1.3 Separate Sets of T-Extendable, A-Extendable, C-Extendable and G-Extendable Primers

### 1.3.1 Extension-Cutback Architectures

Extension-cut back approaches take complexes between the primer and the reference DNA that is bound to with a 3′-underhang, however it is generated, and add nucleoside triphosphates to them in a way that addition of T (for example) renders those primers that added T physically distinct from those that added the other three nucleotides. This physical distinction allows those that added T to be separated from the others. Then, in a separate step on the collection of extension primers that added T, the added T is removed (“cutback”) to leave a 3′-end that, if templated again on the reference DNA, would add T again. These are the T-extendable primers. Of course, this is then repeated with A, G, and C to get the A-extendable. G-extendable”, and C-extendable sets of primers.

Many architectures can be used to implement this. For example, involving both synthetic DNA and processing of natural DNA, a sequence of steps involving the addition of N versus 3N followed by separation (which may not be necessary if the 3N extension products are rendered irreversibly inactive, for example by the addition of the 3N 2′,3′-dideoxynucleotides), requires the cutback of the added N to create a primer that can again add N when it is presented to the target genome.

Many procedures known in the art can be used to create the physical distinction. Preferred are cases where the TTP (the extension nucleotide, in this example) carries a biotin tag, while the 3NTPs (the others) do not. Presently preferred is to therefore have all of the triphosphates be 2′,3′-dideoxynucleoside triphosphates, so that only a single nucleotide is added.

More problematic are procedures that permit the cutting back of the nucleotide added, to regenerate a 3′-end of a primer that is (again, in this case) T-extendable. Four processes are presented.

1.3.2 Using 5′-amino-2′,5′-dideoxynucleoside triphosphates (Example 2)

Presently preferred is a process that introduces the extension nucleoside in its 5′-deoxy-5′-amino-5′-triphosphate, with the remaining 3NTPs in their 2′,3′-dideoxynucleoside forms and optionally carrying a tag that is separable (biotin, thiol). The 5′-aminotriphosphates are prepared by the procedure of [Wol04], which is incorporated herein by reference. In this procedure, the modified nucleosides are prepared in high yields from naturally occurring 2′-deoxynucleosides by tosylation followed by azide replacement and Staudinger reduction. Efficient conversion of these 5′-amino nucleosides to corresponding 5′-N-triphosphate nucleotides was achieved via a one-step reaction with trimetaphosphate in Tris-buffered aqueous solution.

In the extension reaction, if T is called for by the template, a 5′-amino-T is appended to the 3′-end of the primer. Primer extension occurs until it is terminated through the incorporation of a dideoxynucleotide, immediately if a T is not called for. Treatment with dilute acid cleaves any DNA added to regenerate a T-extendable end. Incorporation of sequential T's leads to the same T-extendable primer as incorporation of just one. Primers terminated immediately with a dideoxynucleotide are rendered inactive in all subsequent experiments. They may be, but need not be, removed using a tag that they may (or may not) carry.

1.3.3 Cutback when N is a Ribonucleoside

Both Joyce [Ast98][Joy97] and Patel and Loeb [Pat00] have described mutant Family A polymerases that add a ribonucleotide to the 3′-end of a primer. Ribonucleosides are added to the 3′-end of a DNA primer in a template-directed fashion by T7 RNA polymerase as well. When a set of primers (for example, derived in a solution library) is extended using the reference genome as the template (the reference genome being denatured by heating; it may also be fragmented), the ribonucleosides triphosphate for N, and the 2′-3′-dideoxyribonucleoside triphosphates for 3N, to generate the primer set for N, then all primers that added 3N are irreversibly terminated, while those that added N are terminated in a ribonucleoside or, if multiple additions ensued, by one or more N ribonucleosides eventually terminated in a dideoxyribonucleoside. If multiple additions ensued, treatment with ribonuclease A (RNase A) renders the primers that added N initially in the form where they have been extended by a single N-bearing ribonucleotide.

Treatment of this extended primer bearing a 3′-terminal N-ribonucleotide with sodium periodate at room temperature at neutral pH (the reaction is complete at 10 mM periodate in less than a minute) generates the 2′,3′-dialdehyde, which can be captured by imine formation, separating the primers that were extended through the addition of N from those that were extended through the addition of 3N, through the formation of an imine (for example) with a resin-bound amine, or as an oxime with a resin-bound O-alkoxylamine, or as a hydrazone using a resin-bound hydrazine. Then, using reactions known in the art [Bro53][Whi53], the ketone can be treated to suffer beta-elimination, releasing the original primer with a 3′-O-phosphate. Treatment of this mixture by alkaline phosphatase (resin bound, at pH 8) re-generates an extendable primer with the free 3′-OH. When done on the library, the product is a set of N extendable primers.

This cutback sequence can be used regardless of whether the primers are derived from chemical synthesis, or by fragmentation of the reference genome, or by 3′-exonuclease digestion, or by any other method.

1.3.4 Cutback when N is Preceded by a Ribonucleoside

When synthetic primers are used, or when messenger RNA is used as the source of reference material, the primers have a ribonucleotide already at their 3′-end. Addition of N as its alpha phosphorothioate nucleoside triphosphate, while addition of 3N as its 2′,3′-dideoxynucleoside triphosphate, permits a cutback process that works when N is added but not when 3N is added. This extension may be done by T7 RNA polymerase or, more preferably, by one of the DNA polymerases that accepts a 3′-ribonucleotide in its primer (e.g. Bst DNA polymerase, large fragment, Therminator, T7 DNA polymerase, T4 DNA polymerase, Klenow fragment, or phi29 DNA polymerase). This is based on the fact [Gis88] that treatment of a phosphorothioate that is preceded by a ribonucleosides with iodine (as an oxidizing agent) or with an alkylating agent (such as iodoethane) causes the cleavage via a 2′,3′-cyclic phosphate intermediate. The 3′-end of the primer is then restored by treatment with RNase A (which opens the 2′,3′-cyclic phosphate) followed by alkaline phosphatase.

### 1.3.5 Ligation-Extension Tools

An innovative approach that is our second most preferred approach begins with the fragmented duplex reference DNA, preferably short fragments (most preferably less than 50). The ends of these fragments are polished, and then blunt end ligated to a short duplex that is designed to create a different restriction site depending on what nucleotide is at the 3′-end of the polished duplex. The restriction sites are then used to cut back to reveal one of four extendable ends with the different restriction endonucleases. This is exemplified in Example 3.

### 1.3.6 Exploiting Capture Tags in the Generation of Extendable Primer Sets

In various of the architectures that implement the process of the instant invention, capture tags may be used. These may be used as a part of a required separation; separation is required when the 3N primers are not rendered permanently inactive in the generation of the N primer set. Alternatively, separation may be convenient to remove the 3N-extended primers even if they have been rendered permanently inactive, just to simplify downstream processing by not having a substantial amount of unuseful DNA present.

In each of these cases, it is possible to replace the 2′,3′-dideoxynucleoside triphosphates by the commercially available 2′,3′-dideoxynucleoside triphosphates having a biotinylated capture tag, or the 2′,3′-dideoxynucleoside triphosphates having an alpha thiophosphodiester unit. This allows the primers that have been extended by a 3N triphosphate to be captured on an avidin or mercury column/beads (respectively). Alternatively, the N nucleotide added may carry the capture tag.

### 2. Step 2. Annealing Primers to Complementary Sites in a Target DNA Sample

The second step of the inventive process presents, separately, the T-extendable primer sets, the A-extendable primer sets, the C-extendable primer sets, and the G-extendable primer sets, to the reference DNA, and achieves binding. Procedures to do this that involve heating and cooling are well known in the art.

### 3. Step 3. Using Sets of Primers to Discover Variation at the Queried Site

The third step applies procedures that deliver four mixtures of DNA fragments that are depleted in those that added T, A, C, and G respectively (that is, that added N) and enriched in those that added not-T, not-A, not-C, and not-G respectively (that is, that added 3N). The extracted products are enriched in those that have discovered variation, a difference between the target and reference DNAs.

Again, many architectures may achieve this end. Fundamentally, they involve the addition of N and 3N that differ in a feature that allows them to be differentially separated or differentially processed downstream. This feature can be a tag on the N nucleotide (a biotin, a thiol) that is not present in the 3N nucleotides, or vice versa, where the tag is used to separate the products that have been extended by a 3N opposite the query site (and therefore have discovered variation at the query site). The challenge then is to manage primer extension so that nucleotide addition downstream from the initial addition does not confuse the separation by adding tags where they are not desired.

3.1 Exploiting Irreversibly Terminated Nucleoside Triphosphates for N in Competition with Reversibly Terminated Nucleoside Triphosphates for 3N (Example 3)

This can, in principle, be done with terminators that stop extension after the tagged nucleoside is added. In this case, each of the 3N nucleoside triphosphates are standard and bear a tag, while the N nucleoside triphosphate is not tagged and is presented in a 2′,3′-dideoxy form, thereby irreversibly terminating the extension of primers that did not discover variation at the queried site before they can add a tag by incorporation of a tagged 3N triphosphate downstream from the queried site. Depending on how much template is present, this may ultimately add a 2′,3′-dideoxy-N, however, limiting the options for further analysis of the primers that have discovered variation at the queried site.

The presently preferred way to manage this is to present N as an irreversibly terminating 2′,3′-dideoxynucleoside triphosphate and the 3N triphosphates as standard 2′-deoxynucleoside carrying a 3′-ONH₂ group with a 3′-reversible terminator. For example, the 3′-O-allyl-2′-deoxynucleoside triphosphates are incorporated by THERMINATOR® polymerase and its mutant forms and serve as reversible terminators, blocking extension until it is cleaved with a palladium catalyst [Seo05]. More preferably, the 3′-O—NH₂-2′-deoxynucleoside triphosphates is incorporated with the Tabor-Richardson variant of the Taq DNA polymerase. It also blocks elongation, until it is removed by treating with acetate buffered sodium nitrite:HONO, preferably between pH 6 and pH 7, at room temperature, incubation preferably for less than 30 min. These are conditions where the N-extended sequences remain inert to further extension. Thus, after the terminating triphosphates are removed or destroyed, the 3N-extended sequences can be further extended on the template from the target genome, or ligated to another DNA sequence, which may be used to enter the 454 sequencing procedure, or used for PCR amplification.

A further advantage of using the 3′-ONH2 reversible terminator is that it can be recovered by capture on an immobilized aldehyde. While this recovery is not necessary, since the N-extended primers are no longer active, this separation will further enrich the delivered pool in species that have discovered variation.

For example, it is possible to deliver the output from the 3N-extended primers directly for 454 sequencing. In this case, the output is polished to be double stranded, blunt ended, with all four ends chemically suited for ligation. Downstream 454 sequencing is particularly preferred when the output contains single exemplars of the sequences that have found variation at the queried site.

### 3.2 Exploiting Differentially Terminating Nucleoside Triphosphates for N and for 3N

Different functionality on the 3′-position of the 3N-extended and the N-extended products may also be used to differentially deliver DNA fragments that have discovered a SNP. For example, one architecture presents N as its 2′,3′-dideoxynucleoside triphosphate and 3N as their 2′,3′-dideoxy-3′-aminonucleoside triphosphates. These are incorporated by polymerases known in the art [Tab95], with termination in both cases. Then, the 3′-amino group in the 3N-extended primers can be used to capture a downstream PCR primer binding site, a defined sequence that has a 5′-homologated (a DNA molecule that, at its 5′-end, has the 5′-OH group replaced by a CH₂CHO unit) or, preferentially, a 5′-bishomologated nucleoside (a DNA molecule that, at its 5′-end, has the 5′-OH group replaced by a —CH₂CH₂CHO unit) at its 5′-terminus. These form imines with the 3′-amino group of the 3N-extended primers that can be captured as the secondary amine through treatment with sodium cyanoborohydride at pH 6-8 in a process well known in the art. The downstream PCR primer binding site can be used to amplify the 3N-extended primers, to prepare them for sequencing. Many polymerases, including Taq and Therminator, read through this single unnatural secondary amine linkage in a template.

Alternatively, the homologated or bishomologated species may capture sequence that forms a hairpin. Especially in bead-bound libraries, these can be delivered directly to an Intelligent BioSystems instrument for sequencing.

### 3.3 Exploiting Differential Capture

Through the differential tagging of the N and 3N triphosphates, the 3N-extended and the N-extended products may be separated. For example, if the primer sets have a 3′-ribonucleosides, presenting the 3N-triphosphates as 2′,3′-dideoxynucleosides in a biotinylated form, but not having the N-2′,3′-dideoxynucleoside triphosphates biotinylated, the N-extended and the 3N-extended products may be separated on an avidin column. Then, for downstream sequencing, RNase cleavage will remove the 2′,3′-dideoxynucleoside tag, re-generating a ligatable 3′-terminus (necessary for the 454 sequencing pipeline).

### 3.4 Exploiting Differential Extendability

If the 3N-triphosphates may be presented as ribonucleosides triphosphates using the Joyce polymerase [Ast98], with the N-triphosphate presented as its 2′,3′-dideoxynucleoside, a single extension is achieved, with further extension possible by changing the polymerase to one that accepts a template having a ribonucleoside at its 3′-end.

### Step 4. Determining the Locus of the Variation

In all cases, the output of the third step is a collection of oligonucleotides enriched in those that have found a SNP, or enriched in DNA that can be downstream processed. The preferred form of that output depends on how, downstream, the information in that fragment will be used to place the SNP within the in silico genome.

In many architectures for downstream sequencing, including the 454 architecture, is possible that the downstream sequence will be determined by ligation of a sequencing primer to the 3′-end. If single molecules are delivered, then PCR amplification is desired. If, however, the fragments that have discovered variation are present on a bead made via split-and-pool, with enough copies to be directly sequenced.

### EXAMPLES

### Example 1

### The Ligate-Cleave Procedure to Generate Primer Sets

In addition to creating primer sets by synthesis of specific DNA sequences or by sorting DNA of random sequence, primers can be generated from the reference genome DNA itself (the physical DNA). This includes shearing the physical DNA (by sonication or focused sonication), by restriction endonucleases, by cutting back with an exonuclease, ITCHY technologies, or other ways of creating truncated fragment libraries that are well known in the art.

This example illustrates the use of blunt end ligation followed by restriction endonuclease fragmentation to give, with three known restriction endonuclease, three of the fragments. First, the DNA from the reference genome is fragmented to fragments that are preferably 50-1000 nucleotides in length. The Covaris instrument in the art is known to provide such lengths, with shorter lengths arising from longer Covaris treatment. The ends of the fragments are made blunt ended (“polished”) by treatment with an exonuclease and, more preferably, by filling in with a DNA polymerase and 2′-deoxynucleoside triphosphates. The result is a collection of DNA duplex sequence fragments, with both ends being blunt and having, at each end, one of these four types of blunt end:

| | | | |
|---|---|---|---|
| DNA-T-3′ | DNA-C-3′ | DNA-G-3′ | DNA-A-3′ |
| DNA-A-5′ | DNA-G-5′ | DNA-C-5′ | DNA-T-5′ |

Where the DNA sequences are complementary. The following duplex sequence, prepared by Integrated DNA Technologies (IDT, Coralville) is then attached by blunt-end ligation to each end:

ATCNNNNN-3′-biotin
TAGNNNNN-5′

where N is any nucleotide (but the N's paired in the duplex are, of course, Watson-Crick complementary), and the oligonucleotide segment is as long as needed to get the ligation to go efficiently, preferably at least 10 nucleotides in length, with a sequence chosen to avoid Mbo1 sites and/or to facilitate downstream cloning. This gives the following products.

| | |
|---|---|
| (a) | DNA-TATCNNNNN-3′-biotin |
| | DNA-ATAGNNNNN-5′ |
| (b) | DNA-CATCNNNNN-3′-biotin |
| | DNA-GTAGNNNNN-5′ |
| (c) | DNA-GATCNNNNN-3′-biotin |
| | DNA-CTAGNNNNN-5′ |
| (d) | DNA-AATCNNNNN-3′-biotin |
| | DNA-TTAGNNNNN-5′ |

This creates a restriction site in only case (b); the sequence GATC is the recognition sequence for the restriction enzyme Mbo1. All of the other fragments do not contain sites for this enzyme. Digestion of all possible structures with Mbo1 removes the biotin only from the top strand of (c) to give as the only fragments that do not retain their ability to bind to streptavidin, in the following form:

DNA-DNA-CTAG

Because of the specificity of the Mbo1 restriction endonuclease, the DNA fragments that do not bind to streptavidin, when templated on the reference genome, will all add G first. Analogous separation can be done using other tags (for example, a thiol group at the 3′-will allow a mercury gel or column to separate the non-cleaved fragments from the cleaved fragments). Thus, the DNA fragments recovered from a streptavidin separation step is a set of G-extendable primers from this reference genome. It should be noted that to have utility, this process need not identify every G-extendable primer within the reference genome. Even as little as 20% coverage of the non-repeating regions has utility, more preferably 50% coverage of the non-repeat regions.

The same process is repeated to generate the A-extendable primer set, but with Tsp509I as the restriction endonuclease, with blunt end ligation to the following sequence where the length of the N region and its sequence is chosen as before:

ATTNNNNN-3′-biotin
TAANNNNN-5′

| | |
|---|---|
| (a) | DNA-TATTNNNNN-3′-biotin |
| | DNA-ATAANNNNN-5′ |
| (b) | DNA-CATTNNNNN-3′-biotin |
| | DNA-GTAANNNNN-5′ |
| (c) | DNA-GATTNNNNN-3′-biotin |
| | DNA-CTAANNNNN-5′ |
| (d) | DNA-AATTNNNNN-3′-biotin |
| | DNA-TTAANNNNN-5′ |

This creates a restriction site in only one case, the one that is underlined, for the four cutter Tsp509I. All of the other fragments do not contain sites for this enzyme. Digestion with Tsp509I releases the biotin from (d) to generate:

DNA-DNA-TTAA

Now, the only fragments that do not retain their ability to bind to streptavidin (after the duplex is denatured) will be extended by A when templated on the reference genome. This is therefore a set of A-extendable primers.

The C-extendable primer set is then prepared from the reference genome using StyD41, with blunt end ligation to the following sequence (N's defined as before):

CNGGNNNNN-3′-biotin
GNCCNNNNN-5′

Again where N is any nucleotide. The products are as follows:

| | | |
|---|---|---|
| (a) | DNA-TCNGGNNNNN-3′-biot | SEQ ID 1 |
| | DNA-AGNCCNNNNN-5′ | SEQ ID 3 |
| (b) | DNA-CCNGGNNNNN-3′-biot | SEQ ID 2 |
| | DNA-GGNCCNNNNN-5′ | SEQ ID 4 |
| (c) | DNA-GCNGGNNNNN-3′-biot | SEQ ID 5 |
| | DNA-CGNCCNNNNN-5′ | SEQ ID 7 |
| (d) | DNA-ACNGGNNNNN-3′-biot | SEQ ID 6 |
| | DNA-TGNCCNNNNN-5′ | SEQ ID 8 |

This creates a restriction site in only one case, the one that is underlined, for the four cutter StyD41. All of the other fragments do not contain sites for this enzyme. Digestion with StyD41 gives the following unlabeled fragments that do not bind streptavidin, and which collectively make a C-extendable primers C:

DNA-DNA′GGNCC

No restriction endonuclease is commercially available to create a T-extendable primer set. Nevertheless, these can be obtained upon recognizing that a T-extendable primer will be derived from one of four classes of blunt ends:

| | |
|---|---|
| (a) | DNA-AT-3′ |
| | DNA-TA-5′ |
| (b) | DNA-TT-3′ |
| | DNA-AA-5′ |
| (c) | DNA-GT-3′ |
| | DNA-CA-5′ |
| (d) | DNA-CT-3′ |
| | DNA-GA-5′ |

To generate the T-extendable primers, the fragment ligated must generate a restriction endonuclease site that cuts between the last and next to last sites. Thus, for (b), blunt end ligation of (b) to the segment (left) (N's defined as above) creates the product (middle) with the TTAA recognition site for the MseI enzyme (T*TAA, where * indicates the site of cleavage) generating a T-extendable fragment (right) following MseI cleavage.

| | | |
|---|---|---|
| AANNNNN-3′-biotin | DNA-TTAANNNNN-3′-biotin | DNA-T-3′ |
| AANNNNN-5′ | DNA-AATTNNNNN-5′ | DNA-AAT-5′ |

For case (c), the ApaL1 enzyme is used (with the recognition sequence G*TGCAC) with the duplex for blunt end ligation, the product of that ligation, and the product following cleavage with ApaL shown below:

| | | |
|---|---|---|
| GCACNNN-3′-biotin | DNA-GTGCACNNN-3′-biotin | DNA-G-3′ |
| CGTGNNN-5′ | DNA-CACGTGNNN-5′ | DNA-CACGT-5′ |

For case (d), the AflII enzyme is used (with the recognition sequence C*TTAAG) with the duplex for blunt end ligation, the product of that ligation, and the product following cleavage with AflII shown below:

| | | |
|---|---|---|
| TAAGNNN-3′-biotin | DNA-CTTAAGNNN-3′-biotin | DNA-C-3′ |
| ATTCNNN-5′ | DNA-GAATTCNNN-5′ | DNA-GAATT-5′ |

As is appreciated by one of ordinary skill in the art, other restriction endonucleases exist that can be substituted for the enzymes listed above to generate the same outcome. These may be preferable depending on the methylation of the reference genome. Further, to prevent concatenation in the blunt end ligation, it is preferred that the synthetic short ligation fragments be blocked at their 3′-ends by a dideoxynucleotide. It should be noted that if this is done, the four N-extendable primer sets can be prepared without an absolute need for a separation, as the fragments that are not cleaved to not have a polymerase-active 3′-end. Thus, they cannot interfere with the second step in the process of the instant invention. No restriction endonuclease is commercially available to create the T-extendable primer sets.

### Example 2

### Use of 3′-deoxy-3′-aminonucleoside Triphosphates and Reversible Terminators to Cap Some and Reversibly Block Other Primers

The following experimental procedure exploits 3′-amino 2′,3′-dideoxy triphosphates as well as a variant of the DNA polymerase from Thermus aquaticus (Taq), where the following amino acid have been replaced: E520G, K40I, L616A, and used the 3′-ONH₂ reversible terminators as well. For this, competition studies using Taq475 and a combination of the 3′-amino dideoxy triphosphates and the reversible terminator were used in extension, cleavage and subsequent extension reactions.

### Oligonucleotides Used:

Testing 3′-amino Dideoxy Triphosphates and Reversible Terminator

Primer:
dhSSP1

SEQ ID 9

5′-GCGTAATACG ACTCACTATG GACG-3′
Templates:
Template A

SEQ ID 10

5′-GTCTTCGTGT AACGTCCATA GTGAGTCGTA TTACGC
Template G

SEQ ID 11

5′-GTCTTCGTGT GGCGTCCATA GTGAGTCGTA TTACGC
Template T

SEQ ID 12

5′-GTCTTCGTGT TTCGTCCATA GTGAGTCGTA TTACGC
Template C

SEQ ID 13

5′-GTCTTCGTGT CCCGTCCATA GTGAGTCGTA TTACGC

### For Competition Studies

Primer:
dhSSP1

SEQ ID 14

5′-GCGTAATACG ACTCACTATG GACG-3′
Template:
SNPT1 (A)

SEQ ID 15

5′-GTCTTCGTGT CACGTCCATA GTGAGTCGTA TTACGC-Biot

In a 10 μL reaction volume γ³²P-labeled primer (dh-SS P-1) (0.5 pmol), cold primer (2 pmol) and Template (Template A, G, T, or C) (3 pmol) were annealed by incubation at 96° C. for 5 min and cooled to room temperature. Taq475 (0.25 μg) was added and incubated at 37° C. for 30 sec. Assays contained 20 mM Tris-HCl pH 8.8, 10 mM KCl, 10 mM (NH₄)₂SO₄, 2 mM MgSO₄, and 0.1% Triton X-100. Assays were initiated by triphosphate (Refer to FIG. 1) (100 μM final) and incubated at 37° C. for 2 min. Reactions were then quenched with 10 μL of 10 mM EDTA in formamide with Bromphenol Blue and Xylene Cyanol (both at 1 mg/mL). Samples (6 μL) were resolved on a 16% denaturing polyacrylamide gel and analyzed with a Molecular Imager.

Competition Studies. Steps in this example.

### 1. Immobilization of Primer/Template to Dynabeads via Biotin-Streptavidin Interaction

2. Primer Extension using a combination of reversible and irreversible terminators

### 3. Cleavage Reaction

4. 2^{nd} Primer Extension with dNTPs for full length product

In a 10 μL reaction volume γ³²P-labeled primer (dh-SS P-1) (0.5 pmol), cold primer (2 pmol) and 3′-Biontiylated Template SNPT1 (3 pmol) was annealed by incubation at 96° C. for 5 min and cooled to room temperature. The primer template complex was then immobilized to Streptavidin magnetic beads (Dynabeads) using a 2× binding buffer supplemented with hydroxylamine. Assays contained 20 mM Tris-HCl pH 8.8, 10 mM KCl, 10 mM (NH₄)₂SO₄, 2 mM MgSO₄, and 0.1% Triton X-100 and 2% hydroxylamine. Taq475 (0.25 μg) was added to the reactions and incubated at 37° C. for 30 sec. Four different sets of assays were performed and initiated with various combinations of reversible and irreversible terminators including 1) TTP-ONH₂, 3′amino dd C, G and ATP; 2) CTP-ONH₂, 3′amino ddT, G and ATP; 3) GTP-ONH₂, 3′amino dd C, T and ATP; or 4) ATP-ONH₂, 3′amino dd G, T, and CTP. Each set of triphosphates was added at 100 μM final concentration and incubated at 37° C. for 2 min. Reactions were then quenched with 5 μL of 10 mM EDTA. Samples were washed using the biotin-streptavidin handle to remove residual triphosphate, polymerase and hydroxylamine. Reactions were then treated with cleavage buffer (HONO/dioxane) to remove the 3′-ONH₂. Final extension reactions used dNTPs at 100 μM to generate full length product. DNA was then removed from the biotin-streptavidin handle by heating. Samples (4 μL) were resolved on a 20% denaturing polyacrylamide gel and analyzed with a Molecular Imager.

### Example 3

### Generating Primers by Ligation with SAMRS

### Ligation Reactions Using SAMRS Primers

### Oligonucleotides Used:

11 mer Standard

SEQ ID 16

5′-ATTGTCCGCGG
11 mer SAMRS 6 + 4* + 1

SEQ ID 17

5′-ATTGTCC*G*C*G*G
14 mer Standard

SEQ ID 18

5′-/phos/TCACAGAGAGAGCA/phos/
14 mer SAMRS 5 + 8* + 1:

SEQ ID 19

5′-/phos/TCACAG*A*G*A*G*A*G*C*A/phos/
25 mer Standard

SEQ ID 20

5′-ATTGTCCGCGGTCACAGAGAGAGCA
25 mer SAMRS 16 + 8* + 1

SEQ ID 21

5′-ATTGTCCGCGGTCACAG*A*G*A*G*A*G*C*A
25 mer SAMRS 6 + 4* + 6 + 8*

SEQ ID 22

5′-ATTGTCC*G*C*G*GTCACAG*A*G*A*G*A*G*C*A
Template (52 mer): PAGE purified and Standard
desalted

SEQ ID 23

3′-TA ACAGGCGCCA GTGTCTCTCT CGTTCAACAC CTAGTTATGG
TACCAGAGTC-5′

### Ligation Reactions:

Radioactivity was used to monitor the products: Reactions 1-9 B: cold 11 mer (5′ and 3′ OH) in the ligation and radiolabeled all species after the ligation

| | 1 | 2 | 3 | 4 | 5 | 6 | 7 | 8 | 9 |
|---|---|---|---|---|---|---|---|---|---|
| 11mer Standard Control | + | + | + | | | | | | |
| (+/−32P) [A/B] | | | | | | | | | |
| 11mer Standard/SAMRS | | | | + | + | + | | | |
| 6 + 4* + 1 (+/−32P) [A/B] | | | | | | | | | |
| 14mer Standard Control | | + | | | + | | | | |
| 14mer Standard/SAMRS | | | + | | | + | | | |
| 5 + 8* + 1 | | | | | | | | | |
| 25mer Standard Control | | | | | | | + | | |
| (+/−32P) [A/B] | | | | | | | | | |
| 25mer Standard/SAMRS | | | | | | | | + | |
| Control 16 + 8* + | | | | | | | | | |
| 1 (+/−32P) [A/B] | | | | | | | | | |
| 25mer Standard/SAMRS | | | | | | | | | + |
| Control 6 + 4* + 6 + | | | | | | | | | |
| 8* (+/−32P) [A/B] | | | | | | | | | |
| Template (PAGE purified) | + | + | + | + | + | + | + | + | + |

Ligation 1-9 B reactions used 11mer and 25mers with a 5′ and 3′ hydroxyl group and a 14mer with a 5′ and 3′ phosphate. The reactions were radiolabeled after the ligation. This method is a little more labor intensive and is not as sensitive as when using radiolabeled oligo's during the ligation however it does give cleaner results.

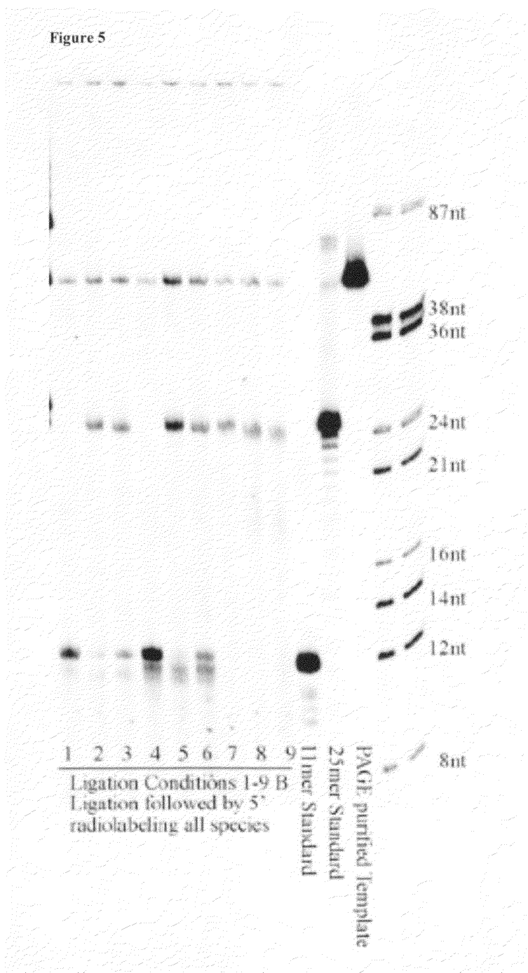

Ligation products (25mers) were seen in reactions 2B, 3B, 5B, and 6B (FIG. 5). These results show that SAMRS containing primers can be used as substrates in ligation reactions using T4 DNA ligase.

### REFERENCES

[Ast98] Astatke, M., Ng, K., Grindley, N. D., Joyce, C. M. (1998) A single side chain prevents Escherichia coli DNA polymerase I (Klenow fragment) from incorporating ribonucleotides. Proc. Natl. Acad. Sci. USA 95, 3402-3407.
[Ben04] Benner, S. A. (2004) Understanding nucleic acids using synthetic chemistry. Acc. Chem. Res. 37, 784-797
[Bro53] Brown, D. M., Fried, M., Todd, A. R. (1953) The determination of nucleotide sequence in polyribonucleotides. Chem. Ind. (London) 352-353
[Fah01] Faham, M., Baharloo, S., Tomitaka, S., DeYoung, J., Freimer, N. B. (2001) Mismatch repair detection (MRD): high throughput scanning for DNA variations. Human Mol. Genetics. 10, 1657-1664
[Fah05] Faham, M., Zheng, J. B., Moorhead, M., Fakhrai-Rad, H., Namsaraev, E., Wong, K., Wang, Z. Y., Chow, S. G., Lee, L., Suyenaga, K., Reichert, J., Boudreau, A., Eberle, J., Bruckner, C., Jain, M., Karlin-Neumann, G., Jones, H. B., Willis, T. D., Buxbaum, J. D., Davis, R. W. (2005) Multiplexed variation scanning for 1,000 amplicons in hundreds of patients using mismatch repair detection (MRD) on tag arrays. Proc. Natl. Acad. Sci. USA 102, 14717-14722
[Fak04] Fakhrai-Rad, H., Zheng, J. B., Willis, T. D., et al. (2004) SNP discovery in pooled samples with mismatch repair detection. Genome Res. 14, 1404-1412
[Gis88] Gish, G., Eckstein, F. (1988) DNA and RNA sequence determination based on phosphorothioate chemistry. Science 240, 1520-1522.
[Joy97] Joyce, C. M. (1997) A single side chain prevents Escherichia coli DNA polymerase I (Klenow fragment) from incorporating ribonucleotides. Proc. Natl. Acad. Sci. USA 94, 1619-1622
[Kim08] Kim, H-J., Kim, M-J., Karalkar, N., Hutter, D., Benner, S. A. (2008) Synthesis of pyrophosphates for in vitro selection of catalytic RNA molecules. Nucleosides, Nucleotides and Nucleic Acids 27, 43-56
[Pat00] Patel, P. H, Loeb, L. A. (2000) Multiple amino acid substitutions allow DNA polymerases to synthesize RNA. Proc. Natl. Acad. Sci. 275, 40266-40272
[Pet07] Peters, B. A., Kan, Z. Y., Sebisanovic, D., et al. (2007) Highly efficient somatic mutation identification using Escherichia coli mismatch-repair detection. Nature Methods 4, 713-715
[Sac01] Sachidanandam, R., Weissman, D., Schmidt, S. C., Kakol, J. M., Stein, L. D., Marth, G., Sherry, S., Mullikin, J. C., Mortimore, B. J., Willey, D. L., et al. 2001. A map of human genome sequence variation containing 1.42 million single nucleotide polymorphisms. Nature 409, 928-933
[Seo05] Seo, T. S., Bai, X., Kim, D. H., Meng, Q., Shi, S., Ruparel, H., Li, Z., Turro, N. J., and Ju, J., (2005) Four-color DNA sequencing by synthesis on a chip using photocleavable fluorescent nucleotides. Proc. Natl. Acad. Sci. 102, 5926-5931
[Sjo08] Sjoblom, T. (2008) Systematic analyses of the cancer genome: lessons learned from sequencing most of the annotated human protein-coding genes. Curr. Opin. Oncol. 20, 66-71
[Tab95] Tabor, S., Richardson, C. C. (1995) A single residue in DNA-polymerases of the Escherichia coli DNA-polymerase I family is critical for distinguishing between deoxyribonucleotides and dideoxyribonucleotides. Proc. Natl. Acad. Sci. USA 92, 6339-6343
[Whi53] Whitfield, P. R., Markham, R. (1953) Natural configuration of the purine nucleotides in ribonucleic acids. Chemical hydrolysis of the dinucleoside phosphates. Nature 171, 1151-1152
[Wol04] Wolfe and Kawate (Wolfe, J. L., Kawate, T. (2004) Synthesis and polymerase incorporation of 5′-amino-2′,5′-dideoxy-5′-N-triphosphate nucleotides. Curr. Protoc. Nucleic Acid Chem. Chapter 13:Unit 13.3),

What is claimed is:

1. A process for generating a collection of oligonucleotides enriched in individual oligonucleotides, each of said individual oligonucleotide binds to a complementary sequence within a target DNA molecule wherein said sequence has a nucleotide replacement at a queried site distinguishing it from an analogous sequence within a reference DNA molecule, wherein said process comprises (i) providing of four sets of primers, called “T-extendable”, “A-extendable”, “C-extendable”, and “G-extendable”, wherein each set, when templated on the reference DNA sequence, is extended (respectively) using a polymerase by thymidine, adenosine, cytidine, or guanidine, (ii) contacting each set separately with target DNA under conditions where the primer can bind to a complementary sequence within the target DNA to form a duplex, and (iii) incubating said duplex with a polymerase to form extended products, wherein the extended products that are formed from T-extendable primers are different if they are extended by T than they are if they are extended by another nucleotide, the extended products that are formed from A-extendable primers are different if they are extended by A than they are if they are extended by another nucleotide, the extended products that are formed from C-extendable primers are different if they are extended by C than they are if they are extended by another nucleotide, and the extended products that are formed from G-extendable primers are different if they are extended by G than they are if they are extended by another nucleotide, and wherein said differences are used to enrich said collection.

2. The process of claim 1, wherein said differences are in the nature of a moiety appended to the 3′-carbon of the 3′-terminal nucleotide.

3. The nucleotide replacements and the flanking sequences wherein variation is found by the process of claim 1.
